SEQUENCE LISTING (1 sequence)

SEQ ID NO: 1 - DNA, 44 bp, Artificial Sequence
  Synthetic
gctcccaccagagtcgtatcttcgaatttccaaacgtaggaaca

(19) United States
(12) Patent Application Publication
(10) Pub. No.: US 2024/0279669 A1
(43) Pub. Date: Aug. 22, 2024

(54) GRAFTED PLANT FOR DELIVERY OF GENOME EDITING REAGENTS

(71) Applicant: Cibus US LLC, San Diego, CA (US)

(72) Inventor: Nathaniel Butler, Roseville, MN (US)

(73) Assignee: Cibus US LLC, San Diego, CA (US)

(21) Appl. No.: 18/248,017

(22) PCT Filed: Oct. 7, 2021
(86) PCT No.: PCT/US2021/053991

§ 371 Date: Apr. 5, 2023

Related U.S. Application Data
(60) Provisional application No. 63/088,800, filed on Oct. 7, 2020.

(51) Int. Cl. C12N 15/82; C12N 9/02; C12N 9/22; C12Q 1/6874; C12Q 1/6895

(52) U.S. Cl. CPC C12N 15/8213; C12N 9/0071; C12N 9/22; C12N 15/8207; C12N 15/8261; C12Q 1/6874; C12Q 1/6895; C12Y 114/19; C12Q 2600/13

(57) ABSTRACT

Embodiments of the present disclosure are directed to a method for producing a grafted plant for delivery of genome editing reagents. The method may include grading cultured rootstock tissue to obtain at least one rootstock expressing an expression construct and having a stem with a graft-compatible diameter and genotype, and making a cut through the at least one rootstock stem and placing a stabilization device adjacent to the cut on the rootstock stem. The method may further include generating a grafted plant by inserting at least one cut scion stem into the stabilization device, wherein the at least one cut scion stem substantially aligns with vascular tissue within the cut rootstock stem, and screening new growth from the grafted plant for gene edits resulting from genomic editing by the expression construct.

### CROSS-REFERENCE TO RELATED APPLICATIONS

This application is a U.S. National Stage Application under 35 U.S.C. § 371 of PCT/US2021/053991 filed Oct. 7, 2021, which claims benefit to Provisional Application No. 63/088,800, filed Oct. 7, 2020, which is incorporated herein by reference in their entireties.

### INCORPORATION-BY-REFERENCE OF MATERIAL SUBMITTED ELECTRONICALLY

The present application contains an electronic sequence listing that was submitted (2024 May 8 C1633106102_Replacement_Sequence_Listing.txt; an ASCII text file; size 473 bytes; and Date of Creation: May 8, 2024), and the contents of which are herein incorporated by reference in its entirety pursuant to 37 C.F.R. § 1.5(e)(5).

### BACKGROUND

Methods of genome editing, which are performed without the use of deoxyribonucleic acid (DNA) for mutation initiation, are referred to as “DNA-free” genome editing techniques. DNA-free genome editing in plants typically requires the direct delivery of transgenic ribonucleic acid (RNA) and/or protein from genome editing reagents to plant cells and regeneration of transformed cells as whole plants and edited lines. This approach requires specialized plant transformation and tissue culture protocols to deliver transgenic RNA and/or protein directly to plant tissues and regenerate transformed material as whole plants. In some cases, RNA viruses have been used to deliver genome editing reagents, such as single guide RNAs (sgRNAs) or small RNAs. However, strict viral size limits prevent larger reagents, such as transcription activator like effector nucleases (TALENs) or Crisper associated protein 9 (Cas9) to be incorporated directly into viral particles. Furthermore, “transient” methods of reagent delivery incorporating protoplasts, Agrobacterium tumefaciens, Rhizobium rhizogenes or geminivirus replicons (GVRs) have also been demonstrated but require costly tissue culture protocol development and risk incorporation of DNA into the host plant. Additionally, some species, such as Cannabis sativis are recalcitrant to direct transformation and regeneration and are therefore, difficult to edit using DNA-free genomic editing techniques.

### SUMMARY

This summary is provided to introduce a selection of concepts in a simplified form that are further described below in the Detailed Description.

In one aspect, the disclosure provides a method for producing a grafted plant for delivery of genome editing reagents. The method comprises grading cultured rootstock tissue to obtain at least one rootstock expressing an expression construct and having a stem with a graft-compatible diameter and genotype and making a cut through the at least one rootstock stem and placing a stabilization device adjacent to the cut on the rootstock stem. The method further includes generating a grafted plant by inserting at least one cut scion stem into the stabilization device, wherein the at least one cut scion stem substantially aligns with vascular tissue within the cut rootstock stem, and screening new growth from the grafted plant for gene edits resulting from genomic editing by the expression construct.

In some examples, the method includes generating a transgenic plant expressing the expression construct in a genotype that is graft-compatible with a genotype of the cut scion stem. In some examples, the method includes generating a transgenic plant expressing the expression construct by infecting a host plant with Agrobacterium tumefaciens carrying the expression construct. In some examples, the method includes including generating a transgenic plant expressing the expression construct by infecting a host plant with Rhizobium rhizogenes carrying the expression construct. In some examples, the method includes generating a transgenic plant expressing the expression construct using particle bombardment. In some examples, the method includes making the cut through the at least one rootstock stem includes making a first angled cut through the at least one rootstock stem, the method further including making a second angled cut through the at least one scion stem, wherein the second angled cut through the at least one scion stem is substantially similar to the first angled cut through the at least one rootstock stem. In some examples, the method includes making the cut through the at least one rootstock stem includes making a first wedge-shaped cut through the at least one rootstock stem, the method further including making a second wedge-shaped cut through the at least one scion stem, wherein the second wedge-shaped cut through the at least one scion stem is substantially similar to the first wedge-shaped cut through the at least one rootstock stem. In some examples, the expression construct includes transcription activator like effector nuclease (TALEN) mRNA, and wherein screening the new growth from the grafted plant includes sampling new shoot growth for the TALEN mRNA and/or protein using end-point reverse transcriptase PCR (RT-PCR) or western blot. In some examples, the expression construct includes an mRNA coding sequence, and a promoter. In some examples, the promoter is 35S. In some examples, the promoter is nopaline synthase (Nos).

In another aspect, the disclosure provides a non-naturally occurring plant, generated by a genomic editing technique. In such embodiments, the genomic editing technique includes generating a transgenic plant by infecting a host plant with Agrobacterium tumefaciens, a Rhizobium rhizogenes solution, or using particle bombardment including a TALEN mRNA coding sequence, a zip-code element from a phloem-mobile RNA, and a constitutive, inducible or phloem-specific promoter. Rhizobium rhizogenes may also be referred to as Agrobacterium rhizogenes (A. rhizogenes), and the terms may be used interchangeably herein. The disclosure further includes grading rootstock tissue of the transgenic plant to obtain at least one rootstock expressing an expression construct and having a stem with a graft-compatible diameter and genotype and making a cut through the at least one rootstock stem and placing a stabilization device adjacent to the cut on the rootstock stem. Moreover, the genomic editing technique includes generating a grafted plant by inserting at least one cut scion stem into the stabilization device, wherein the at least one cut scion stem substantially aligns with vascular tissue within the cut rootstock stem, and screening new growth from the grafted plant for gene edits resulting from genomic editing by the expression construct.

In some examples the genomic editing technique includes making a cut through the at least one rootstock stem and placing a stabilization device adjacent to the cut on the rootstock stem. In some examples, the genomic editing technique includes screening new growth from the grafted plant for gene edits resulting from genomic editing by the expression construct. In some examples, the TALEN mRNA coding sequence targets a Glycine max fatty-acid-desaturase 3 (GmFAD3) gene, a Cannabis sativa phytoene desaturase (CsPDS) gene, or a Solanum tuberosum phytoene desaturase (StPDS) gene.

### DESCRIPTION OF THE DRAWINGS

The foregoing aspects and many of the attendant advantages of this invention will become more readily understood by reference to the following detailed description, when taken in conjunction with the accompanying drawings, wherein:

FIG. 1 illustrates an example method for producing a grafted plant for delivery of genome editing reagents, consistent with the present disclosure.

FIG. 2 is a diagram further illustrating an example method for producing a grafted plant for delivery of genome editing reagents, consistent with the present disclosure.

FIGS. 3A and 3B illustrate example expression constructs for delivery of genome editing reagents, consistent with the present disclosure.

FIGS. 4A, 4B, 4C, and 4D illustrate stages of generating a micrografted potato plant, consistent with the present disclosure.

FIG. 5 illustrates data obtained from various grafting experiments conducted, consistent with the present disclosure.

FIGS. 6A and 6B illustrate transgenic gene expression in the rootstock of a grafted soy plant, consistent with the present disclosure.

FIGS. 7A, 7B, and 7C illustrate transgenic gene expression in the rootstock of a grafted hemp plant, consistent with the present disclosure.

FIGS. 8A, 8B, and 8C illustrate results of genomic editing of wild-type scion tissues in grafted hemp plants, consistent with the present disclosure.

FIGS. 9A, 9B, 9C, 9D, 9E, 9F, 9G, 9H, 9I, and 9J illustrate results of genomic editing of wild-type tissues in grafted soy plants, consistent with the present disclosure.

### DETAILED DESCRIPTION

Various genomic editing methods allow for DNA-free editing in plants. These methods include direct delivery of RNA and/or protein, “transient” DNA delivery via protoplasts, Agrobacterium tumefaciens, Rhizobium rhizogenes, or particle bombardment, and viral delivery of RNA reagents (i.e., sgRNAs and small RNAs) or DNA (i.e., geminivirus replicons). All of these methods require some degree of reagent delivery to the target plant genotype and regeneration of edited tissues. Furthermore, these delivery methods are often inefficient and require preparation of RNA, protein and viral particles which may further reduce efficiencies.

Plant transformation and tissue culture techniques present significant limitations to genome editing, requiring extensive time, labor and materials to develop and implement specialized protocols. DNA-free editing techniques may save time by not requiring incorporation of transgenic DNA.

Plant grafting, referred to herein as “grafting” or to “graft,” refers to or includes a horticultural technique in which the vascular tissue from one plant fuses with the vascular tissue of another plant, such that the two plants form a single grafted plant through the inosculation of their vascular tissue. There are many advantages of grafted plants including, but not limited to, enhanced plant vigor, better disease resistance, improved tolerance to environmental stresses, and heavier crops that are produced over an extended harvest period. Plant grafting may also help plants ward off other infestations, including early blight (Alternaria solani), late blight (Phytophthora infestans), and blossom end-rot (a physiological disorder caused by low calcium levels). Grafted plants may also be more tolerant of environmental stresses like salinity or temperature extremes.

Grafting of transgenic to non-transgenic plant materials for DNA-free genome editing reagent delivery, consistent with the present disclosure, allows for DNA-free genome editing reagents to be delivered to plant tissues without the need for preparing and delivering reagents directly to plant tissues and may expand the number of plant genotypes capable of being edited.

The vascular system of plants allows for the transportation of water and minerals (via the xylem) and sugars (via the phloem) to growing parts of the plant (i.e., sinks). Macromolecules, such as RNA and protein are also transported through the vascular system (primarily the phloem) for long-distance signaling and control multiple plant functions. Long-distance signaling works by the synthesis of macromolecules in the companion cells of the phloem in leaves and roots (i.e., sources), and the loading macromolecules into sieve elements for transport to distant growing parts of the plant (i.e., sinks) via the plasmodesmata. As disclosed herein, expression of genome editing reagents in phloem companion cells may facilitate long-distance transport of transgenic RNA and/or protein and DNA-free editing in sink tissues for propagation and isolation of individual edited events.

Many benefits are achieved by graft delivery of genome editing reagents. First, direct transformation and regeneration of target genotypes is not required for developing editing events in target genotype backgrounds, which reduces the cost of developing and implementing genotype-specific transformation and regeneration protocols. Second, preparation of DNA-free reagents, such as RNA and protein is not required, which further reduces the production cost and technical development of such dedicated protocols. Third, once transgenic lines expressing genome editing reagents have been developed, tissue culture is not required for delivery of the genome editing reagents, and production may be conducted in non-tissue culture environments. Advantages of the present disclosure are not limited to those enumerated above, and additional benefits may be realized.

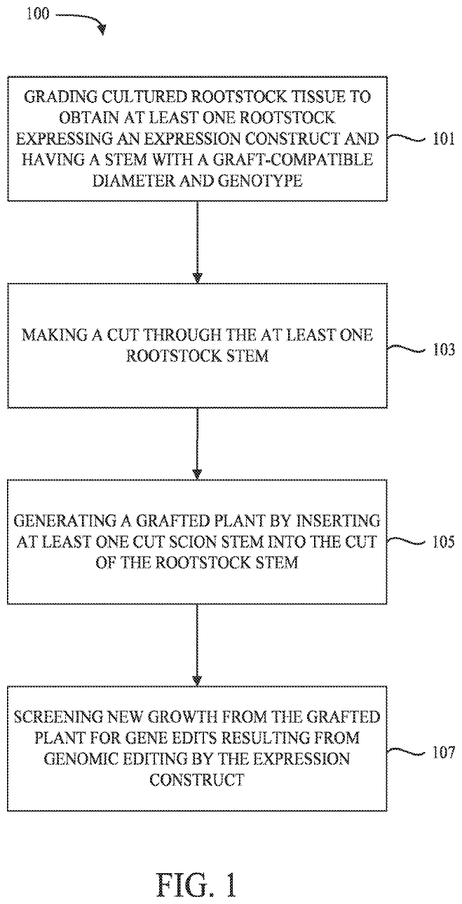

FIG. 1 illustrates an example method 100 for producing a grafted plant for delivery of genome editing reagents, consistent with the present disclosure. At 101, the method 100 includes grading cultured rootstock tissue to obtain at least one rootstock expressing an expression construct and having a stem with a graft-compatible diameter and genotype. As used herein, the term “rootstock” refers to or includes the lower portion of a grafted plant that imparts the roots to the grafted plant. The term “scion” refers to or includes the upper portion of a grafted plant that imparts the leaves, flowers, and/or fruit to the grafted plant. The terms “grade” or “grading” refer to or include a process of assessing or evaluating plants or plant tissue using certain criteria or to identify certain attributes. In the present disclosure, grading cultured rootstock tissue includes assessing or evaluating rootstock tissue for expression of mRNA and/or protein resulting from genomic editing. In various examples, the cultured rootstock tissue may be graded using end-point reverse-transcriptase PCR (RT-PCR) or western blot. In some examples, grading involves identifying rootstock with stems having graft-compatible diameters and genotypes. Graft-compatible stem diameters may range from about 0.5 millimeters (mm) to about 3.0 mm. Graft-compatible genotypes are defined as being sufficiently close in genetic relationship between rootstock and scion for a successful graft union to form, assuming that other factors such as diameter, humidity, and temperature are met.

In some examples, the method 100 begins with generating a transgenic plant expressing the expression construct in a genotype that is graft-compatible with a genotype of the cut scion stem. For instance, transgenic plants expressing TALEN may first be generated in a genotype or species that is graft-compatible with the target genotype using various methods. The method of generating the transgenic plant may depend on the target species. For instance, the method may include generating a transgenic plant expressing the expression construct by infecting a host plant with Agrobacterium tumefaciens carrying the expression construct. GV3101, AGL1, 18r12v, EHA105, LBA4404, MP90 Agrobacterium tumefaciens is an Agrobacterium species used to transform plant cells and that results in the availability of “disarmed strains” that are capable of delivering a single transfer DNA (T-DNA) to plant host tissues without introducing additional T-DNAs used by the bacteria for pathogenesis. Disarmed stains are defined as strains of Agrobacterium tumefaciens that no longer carry so called tumor inducing (Ti) plasmids with additional T-DNAs used for pathogenesis. Infected plant host tissues can be used for regeneration and development of transgenic lines capable of expressing genes on the delivered T-DNA. T-DNAs, such as those from binary vectors carrying genome editing reagents may be delivered to host plant tissues and may be expressed in plant tissues. TALEN is one example of a binary vector carrying genome editing reagents that may be delivered to the plant host tissue, as discussed further therein. Examples are not limited to generating the transgenic plant via infection with Agrobacterium tumefaciens. In additional and/or alternative embodiments, the method 100 may include generating the transgenic plant expressing the expression construct using particle bombardment or Rhizobium rhizogenes.

As used herein, an expression construct refers to or includes a nucleic acid sequence including one or more binary vectors carrying genome editing reagents (such as TALEN mRNA), and a promoter. The term ‘promoter’ refers to or includes a sequence of DNA that turns a gene on or off. In some examples, the promoter may be a constitutive promoter that is active in vivo, an inducible promoter that may be turned on and/or off, or phloem-specific promoter that has activity in phloem tissue. In some examples, the expression construct may include one or more zip-code elements. As used herein, a zip-code element refers to or includes cis-acting signals, or ‘zip-codes’, that permit mRNA sequences to transport through the phloem (e.g., a phloem-mobile RNA). A zip-code element may include an untranslated region (UTR) from the phloem-mobile RNA, or the full-length sequence from a phloem-mobile RNA. Non-limiting examples of a zip-code element include gibberellic acid insensitive (GAI) mRNA such as Arabidopsis GAI, and knotted1-like homeobox (KNOX) mRNA, Tomato KNOTTED1 (LeT6), Potato BEL5, Arabidopsis tRNAmet (At5g57885), Arabidopsis tRNAgly (At5g57885), Arabidopsis CENTRORADIALIS (ATC), Potato KNOTTED1 (StPOTH1), Pumpkin NACP (NAM, ATAF1/3 and CUC2), and Pumpkin GAIP, among others. Various embodiments in accordance with the present disclosure can include at least some of substantially the same features and attributes, including zip-code elements and gene sequences, as discussed in the following references, each of which are hereby incorporated by reference in their entireties for their general teachings related to zip-code elements and the specific teachings related to the sequence(s) of the particular genes: Huang, N C.; Yu, T S. The sequences of Arabidopsis GA-INSENSITIVE RNA constitute the motifs that are necessary and sufficient for RNA long-distance trafficking. Plant J. 2009, 59, 921-929; Kim, M.; Canio, W.; Kessler, S.; Sinha, N. Developmental changes due to long distance movement of a homeobox fusion transcript in tomato. Science 2001, 293, 287-289; Banerjee, A. K.; Chatterjee, M.; Yu, Y.; Suh, S. G.; Miller, W. A.; Hannapel, D. J. Dynamics of a mobile RNA of potato involved in a long-distance signaling pathway. Plant Cell 2006, 18, 3443-3457; Li, C.; Gu, M.; Shi, N.; Zhang, H.; Yang, X.; Osman, T.; Liu, Y.; Wang, H.; Vatish, M.; Jackson, S.; et al. Mobile FT mRNA contributes to the systemic florigen signalling in floral induction. Sci. Rep. 2011, 1, 73; Zhang, W.; Thieme C. J.; Kollwig G.; Apelt, F.; Yang, Lei.; Winter, N.; Andresen, N.; Walther, D.; Kragler, F. tRNA-Related Sequences Trigger Systemic mRNA Transport in Plants. Plant Cell 2016, 28, 1237-1249; Huang, N.C.; Jane, W. N.; Chen, J.; Yu, T. S. Arabidopsis CENTRORADIALIS homologue acts systemically to inhibit floral initiation in Arabidopsis. Plant J. 2012, 72, 175-184; Mahajan, A.; Bhogle, S.; Kang, I. H.; Hannapel, D. J.; Banerjee, A. K. The mRNA of a Knotted1-like transcription factor of potato is phloem mobile. Plant Mol. Biol. 2012, 79, 595-608; Ruiz-Medrano, R.; Xoconostle-Cazares, B.; Lucas, W. J. Phloem long-distance transport of CmNACP mRNA: Implications for supracellular regulation in plants. Development 1999, 126, 4405-4419; Haywood, V.; Yu, T. S.; Huang, N. C.; Lucas, W. J. Phloem long-distance trafficking of GIBBERELLIC ACID-INSENSITIVE RNA regulates leaf development. Plant J. 2005, 42, 49-68.

Also as used herein, a phloem-specific promoter refers to or includes a promoter that targets phloem-specific gene expression. These promoter elements may be associated with genes that are expressed specifically in phloem cells or from organisms that are phloem limited. Non-limiting examples of phloem-specific promoters include sucrose transport protein 1 (SUT1), figwort mosaic virus (FMV), sucrose transport protein 2 (SUC2), Arabidopsis SUC2, Tomato SUT1, Potato PTB1, and Agrobacterium rolC. Also as used herein, a constitutive promoter refers to or includes a promoter that targets phloem-specific gene expression. These promoter elements may be associated with genes that are expressed across plant cell types and may come from non-plant sources. Non-limiting examples of constitutive promoters include 35S promoter, 2×35S promoter, nopaline synthase (Nos) promoter, VaUbi3, among others. Also as used herein, an inducible promoter refers to or includes a promoter that targets phloem-specific gene expression. These promoter elements may be associated with gene expression induced by exposure to an exogenous factor (i.e., β-estradiol) and may come from non-plant sources. Non-limiting examples of inducible promoters include P16ΔS:sXVE promoter, SUPERR:sXVE promoter, among others. Various embodiments in accordance with the present disclosure can include at least some of substantially the same features and attributes, including promoters and gene sequences, as discussed in the following references, each of which are hereby incorporated by reference in their entireties for their general teachings related to plant genetics and the specific teachings related to the sequence(s) of the particular promoters: Srivastava, A. C.; Ganesan, S.; Ismail, I. O.; Ayre, B. G. Functional Characterization of the Arabidopsis AtSUC2 Sucrose/H+ Symporter by Tissue-Specific Complementation Reveals an Essential Role in Phloem Loading but Not in Long-Distance Transport. Plant Physiol. 2008, 148, 200-211; Kühn, C.; Hajirezaei, M. R.; Fernie, A. R., Roessner-Tunali, U.; Czechowski.; Hirner, B.; Frommer, W. B. The Sucrose Transporter StSUT1 Localizes to Sieve Elements in Potato Tuber Phloem and Influences Tuber Physiology and Development. Plant Physiol. 2003, 131, 102-113; Butler, N. M.; Hannapel, D. J. Promoter activity of polypyrimidine tract-binding protein genes of potato responds to environmental cues. Planta 2012, 236, 1747-1755; Schmiilling, T.; Schell, J.; Spena, A. Promoters of the rolA, B, and C Genes of Agrobacterium rhizogenes are Differentially Regulated in Transgenic Plants. Plant Cell 1989, 1, 665-670; Schlückinga, K.; Edel, K. H.; Drerup, M. M.; Köster, P.; Eckert, C.; Steinhorst, L., Waadt, R.; Batistič, O.; Kudla, J. A New β-Estradiol-Inducible Vector Set that Facilitates Easy Construction and Efficient Expression of Transgenes Reveals CBL3-Dependent Cytoplasm to Tonoplast Translocation of CIPK5. Mole. Plant 2013, 6, 1814-1829.

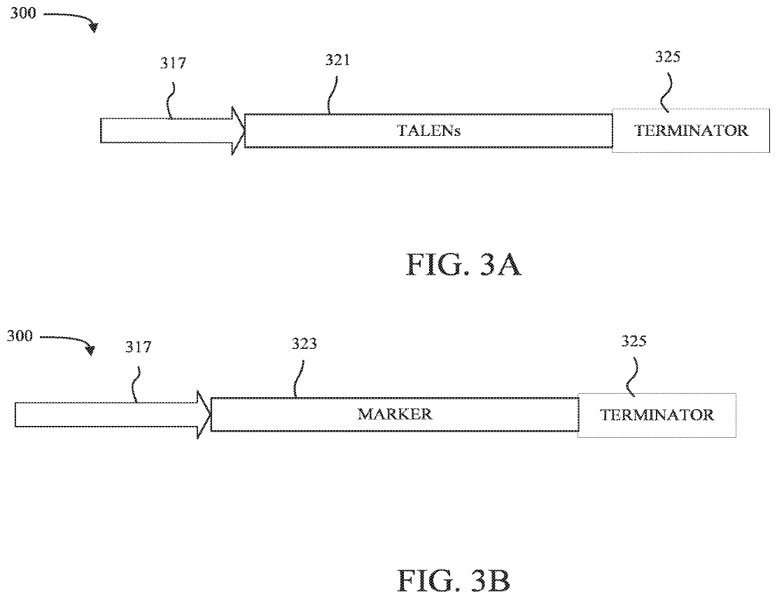

The expression construct may include a variety of nucleic acid segments, selected and arranged to facilitate long-distance transport of genome editing reagents in the phloem of the host plant. For instance, the expression construct may include a TALEN mRNA. In some examples, the expression construct may include an mRNA coding sequence, and a promoter. An example expression construct is illustrated in FIG. 3 and discussed further herein.

Accordingly, the expression construct used to generate the transgenic plants may include a number of components, such as an mRNA coding sequence, and a promoter. Individual transgenic plants may be screened and selected for high expression of the TALEN mRNA and/or protein using end-point reverse-transcriptase PCR (RT-PCR) or western blot. For instance, in some examples, the phloem-mobile RNA included in the expression construct is GAI. In some examples, the phloem-mobile RNA may be KNOX. Similarly, in some examples, the promoter is SUT1. In some examples, the promoter may be SUC2. The promoter is not limited to the particular examples listed. A different promoter may be used, as discussed herein.

As discussed further with regards to FIG. 3, the promoter may be upstream from the mRNA coding sequence. Examples are not so limited, and additional and/or different expression constructs are contemplated.

At 103, the method 100 includes making a cut through the at least one rootstock stem. In some examples, the method 100 includes placing a stabilization device adjacent to the cut on the rootstock stem, though examples are not so limited. The transgenic plant generated at 101 may be grafted to wild-type plants as rootstocks. This may either be done in tissue culture (i.e., micrografting) or in soil conditions (i.e., traditional grafting) depending on the species. Equipment (i.e., choice of stabilization device), type of cut (i.e., wedge vs diagonal), and other grafting techniques may depend on the species and grafting conditions. Various stabilization devices may be used, including but not limited to, tape, plastic wrap, rubber bands, clips, and the like, or any combinations thereof. In some examples, the transgenic/wild-type graft may be created without the use of a stabilization device. For instance, a “V-shape” cut may be made through the transgenic rootstock, and a corresponding “V-shape” cut may be made through the wild-type scion. As discussed further herein, the cut scion may be placed in the corresponding cut rootstock in such a manner that a stabilization device is not needed (e.g., the angle of the cut of the rootstock maintains the scion in place without the use of a stabilization device).

At 105, the method 100 includes generating a grafted plant by inserting at least one cut scion stem into the cut of the rootstock stem, and/or into a stabilization device (if applicable), where the at least one cut scion stem substantially aligns with vascular tissue within the cut rootstock stem. Various types of cuts may be made on the rootstock stem and the scion, and the type of cut made may depend on the species and grafting conditions. For example, making the cut through the at least one rootstock stem may include making a first angled cut through the at least one rootstock stem, and making a second angled cut through the at least one scion stem, where the second angled cut through the at least one scion stem is substantially similar to the first angled cut through the at least one rootstock stem. As a further example, making the cut through the at least one rootstock stem may include making a first wedge-shaped cut through the at least one rootstock stem, and making a second wedge-shaped cut through the at least one scion stem, where the second wedge-shaped cut through the at least one scion stem is substantially similar to the first wedge-shaped cut through the at least one rootstock stem.

At 107, the method 100 includes screening new growth from the grafted plant for gene edits resulting from genomic editing by the expression construct. For instance, grafted plants may be monitored for successful grafting. A successful graft may be indicated by the growth of new shoot tissue (i.e., new meristems, leaves, branches and/or inflorescences) and callus production around the graft junction, respectively. New shoot growth may be sampled for detection of TALEN mRNA and/or protein using end-point reverse transcriptase PCR (RT-PCR) or western blot, respectively. Shoot growth positive for TALEN mRNA and/or protein may be screened for detection of edits using Illumina® amplicon sequencing of the TALEN target gene. Shoot growth positive for edits may be further propagated either vegetatively or through seed to stabilize edits in individual plants, depending on the species. Accordingly, the method 100 may include screening the new growth from the grafted plant for detectable transgenic mRNA or protein from the genomic editing, and propagating tissue from the detectable transgenic mRNA or protein.

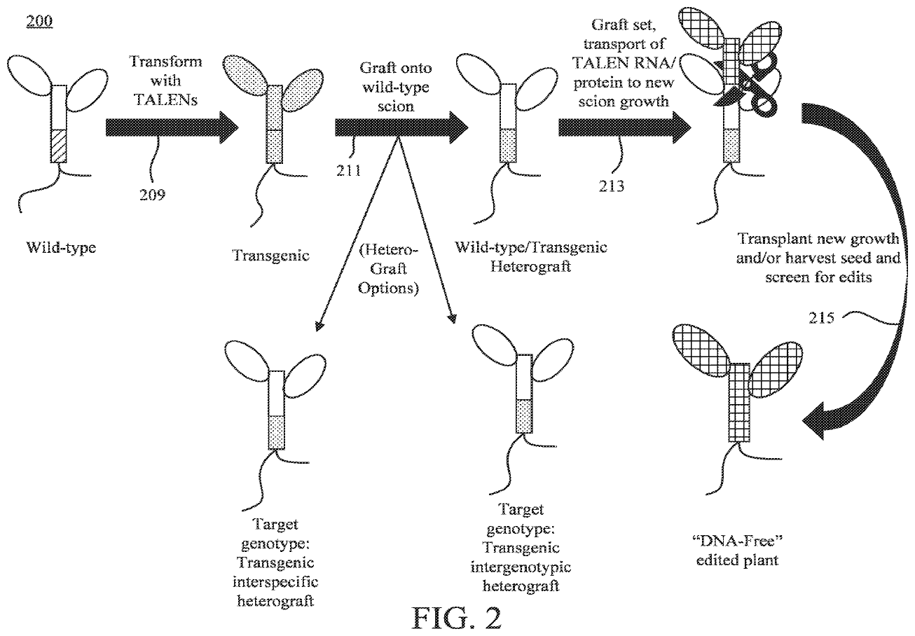

FIG. 2 is a diagram further illustrating an example method 200 for producing a grafted plant for delivery of genome editing reagents, consistent with the present disclosure. In FIG. 2, the method 200 includes at 209, generating a transgenic plant by infecting a host plant with an Agrobacterium tumefaciens solution. In accordance with the present disclosure, an mRNA of interest may be targeted for long-distant transport by positioning the mRNA's coding sequence upstream to a promoter. An example of such mRNA includes TALEN mRNA, and an example promoter to be positioned upstream to the mRNA's coding sequence includes 35S and Nos. Non-limiting examples of phloem-specific promoters include sucrose transport protein 1 (SUT1), figwort mosaic virus (FMV), sucrose transport protein 2 (SUC2), Arabidopsis SUC2, Tomato SUT1, Potato PTB1, and Agrobacterium rolC.

At 211, the method 200 includes grafting the transgenic rootstock onto a wild-type scion. Particularly, as described with regards to FIG. 1, the grafting process may include grading rootstock tissue of the transgenic plant to obtain at least one rootstock expressing an expression construct and having a stem with a graft-compatible diameter. The grafting process may further include making a cut through the at least one rootstock stem, placing a stabilization device adjacent to the cut on the rootstock stem, and generating a grafted plant by inserting at least one cut scion stem into the stabilization device, where the at least one cut scion stem substantially aligns with vascular tissue within the cut rootstock stem. This grafted plant, comprises a wild-type/transgenic heterograft. The heterograft may target a particular plant species, in which the heterograft may be referred to as a transgenic interspecific heterograft. For instance, a difficult species or genotype to genetically modify (such as MN151 variety of soy) may be grafted with a species or genotype that is less difficult to genetically modify (such as Bert variety of soy). In some examples, the heterograft may target a particular genotype, in which case the heterograft may be referred to as a transgenic intergenotypic heterograft. As illustrated in FIG. 2, translocation of transgenic mRNA and/or protein is promoted through the wild-type/transgenic chimeric plant, from the transgenic rootstock to the wild-type scion.

At 213, the method 200 includes transport of TALEN mRNA and/or protein to new scion growth. New shoot growth may be sampled for detection of TALEN mRNA and/or protein using end-point reverse transcriptase PCR (RT-PCR) or western blot, respectively. The degree of editing in the heterograft plant may be directly related to the abundance of transgenic mRNA and/or protein in sink tissues and may be tracked using various methods of mRNA and protein detection. For instance, heterograft plants may be assayed for accumulation of transgenic RNA and/or protein in new shoot tissue (i.e., new leaves, branches and/or inflorescences). New shoot growth may be sampled for detection of TALEN mRNA and/or protein using end-point reverse transcriptase PCR (RT-PCR) or western blot, respectively. Shoot growth positive for TALEN mRNA and/or protein may be screened for detection of edits using Illumina® amplicon sequencing of the TALEN target gene. Accordingly, the method 200 may include screening new growth from the grafted plant for gene edits resulting from genomic editing by the expression construct. At 215, the method 200 may include transplanting shoot growth positive for edits and/or harvesting seed and screening the propagated seed for edits in individual plants, depending on the species.

Various examples of the present disclosure relate to a non-naturally occurring plant generated by the method 100 described with regards to FIG. 1 and/or the method 200 described with regards to FIG. 2. Similarly, the present disclosure relates to a non-naturally occurring seed, reproductive tissue, or vegetative tissue generated by the method 100 described with regards to FIG. 1 and/or the method 200 described with regards to FIG. 2. The present disclosure further relates to a wild-type/transgenic plant generated by the method 100 described with regards to FIG. 1 and/or the method 200 described with regards to FIG. 2. For instance, consistent with methods 100 and 200, a non-naturally occurring plant may be generated by a DNA-free genomic editing technique.

FIG. 3A is a diagram illustrating an example expression construct 300 for delivery of genome editing reagents, consistent with the present disclosure. FIG. 3A illustrates an expression construct 300 comprising an mRNA coding sequence 321, and a promoter 317. Non-limiting examples of mRNA coding sequences used may include a TALEN mRNA sequence targeting the Glycine max fatty-acid-desaturase 3 (GmFAD3) gene, a Cannabis sativa phytoene desaturase (CsPDS) gene, and the Solanum tuberosum phytoene desaturase (StPDS) gene. In some examples, the expression construct 300 may include a zip-code element (not illustrated) from a phloem-mobile RNA. For instance, one or more zip-code elements may be incorporated, upstream from the mRNA coding sequence, downstream from the mRNA coding sequence 321, or both. As may be appreciated, upstream can include a location proximal to and/or closer to the 5′ end of the promoter 317 as compared to the referenced sequence. Conversely, downstream can include a location proximal to and/or closer to the 3′ end of the terminator sequence 325 as compared to the referenced sequence.

As discussed with regards to FIG. 2, the zip-code elements may be from a different phloem-mobile RNA and/or from the same phloem-mobile RNA. Moreover, each of the zip-code elements may be an UTR from the phloem-mobile RNA or full-length sequence. As non-limiting examples, the phloem-mobile RNA is GAI and/or KNOX. As non-limiting examples, the promoter is SUT1 and/or SUC2.

FIG. 3B illustrates an example expression construct 300 including a promoter 317 coupled to a detectable marker 323. Non-limiting examples of a detectable marker include β-glucuronidase (GUS) or florescent protein reporter such as RFP or YFP

Various embodiments are implemented in accordance with the underlying provisional application, U.S. Provisional Application No. 63/088,800, filed on Oct. 7, 2020, and entitled “Grafted Plant for Delivery of Genome Editing Reagents”, to which benefit is claimed and which is fully incorporated herein by reference in entirety. Embodiments discussed in the provisional application is not intended, in any way, to be limiting to the overall technical disclosure, or to any part of the claimed invention unless specifically noted.

Publications cited herein and the subject matter for which they are cited are hereby specifically incorporated by reference in their entireties.

The skilled artisan would recognize that various terminology as used in the Specification (including claims) connote a plain meaning in the art unless otherwise indicated. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

### EXPERIMENTAL/MORE DETAILED EMBODIMENTS

As further illustrated below in connection with the experimental embodiments, genome editing reagents may be delivered via grafted plants. Various embodiments in accordance with the present disclosure can include at least some of substantially the same features and attributes as discussed in the following references, each of which are hereby incorporated by reference in their entireties for their general teachings related to plant genetics and the specific teachings related to the preparation of transgenic plants: Li S, Cong Y, Liu Y, Wang T, Shuai Q, Chen N, Gai J, Li Y. Optimization of Agrobacterium-mediated transformation in soybean. Front. Plant Sci. 2017 February; https://doi.org/10.3389/fpls.2017.00246; Han E H, Goo Y M, Lee M K, Lee S W. An efficient transformation method for a potato (Solanum tuberosum L. var. Atlantic). J Plant Biotechnol. 2015; 42:77-82; Feeney M, Punja ZK. Tissue culture and Agrobacterium-mediated transformation of hemp (Cannabis sativa L.). In vitro Cell. Dev. Biol.—Plant. 2003 November-December; 39:578-585.

Transgenic soy lines were micrografted to wild-type for genome editing, consistent with the following protocol. 2-3 weeks prior to the experiment, wild-type and transgenic soy seed was sterilized by putting the seed in a 9 L desiccator chamber with approximately 100 mL bleach in a 250 ml beaker. Approximately 3.5 mL concentrated HCL was slowly added and the desiccator chamber was kept closed for approximately 16 hours for chlorine gas sterilization. The chlorine gas was allowed to dissipate prior to seed use. Sterilized seeds were transferred to Phytatrays™ with MS (Murashige & Skoog) media by submerging seeds and directing the seed hilum downward. The seeds were placed under 16/8-hour light/dark (75 lumens, approximately 28° C.). Once the seedlings reached the V2 stage (vegetative 2 nodes), cotyledons and fully emerged leaves were removed from all seedlings with a sterile scalpel and aseptic technique. Scion cuttings from the wild-type seedlings were prepared by excising the shoot, abaxial to the second node at a 45-degree diagonal. Rootstock cuttings from the transgenic seedlings were prepared by excising the shoot, abaxial to the cotyledon node at a 45-degree diagonal while keeping the rootstock in the media. A sterile grafting clip or tape matching the diameter of the cut end of the rootstock was placed so that at least 0.5 cm of the clip or tape was overlapping on each side of the cut end. The cut end of the scion was inserted into contact with the cut end of the rootstock using the grafting clip or tape to secure the scion. Grafting stakes were used as needed to secure the scion and maintain direct contact with the rootstock. Phytatrays™ were closed and placed under 16/8-hour light/dark (75 lumens, approximately 28° C.) until callus was formed within the graft and new vegetative growth is seen (graft set: approximately 1-2 weeks). New shoot growth was sampled and propagated in tissue culture or soil to isolate individual genome edited lines.

Transgenic potato and hemp lines were micrografted to wild-type for genome editing, consistent with the following protocol. Approximately 3-4 weeks prior to the experiment, tissue culture wild-type and transgenic plantlets were propagated in Magenta™ boxes with MMS (modified Murashige & Skoog) using plantlet shoot tips and aseptic technique. The plantlets were placed under 16/8-hour light/dark (75 lumens, approximately 23° C.). Once plantlets reached the 5-node stage, fully emerged leaves basal to the 4th node were removed, leaving a single node of fully emerged leaves and the apical meristem. Scion cuttings were prepared from the wild-type plantlets by excising the shoot, abaxial to the 4th node by making 60-degree cuts on either side, creating a spear cut end. Rootstock cuttings were prepared from the transgenic plantlet by excising the shoot, abaxial to the 2nd node by making a vertical cut in the middle of the cut surface, creating a V-cut while keeping the rootstock in the media. A sterile grafting clip or tape matching the diameter of the cut end of the rootstock was placed so that at least 0.5 cm of the clip or tape is overlapping on each side of the cut end. The cut end of the scion was inserted into the cut end of the rootstock and secured using the grafting clip or tape. Grafting stakes were used as needed to secure the scion and maintain direct contact with the rootstock. The Magenta™ boxes were closed and placed under 16/8-hour light/dark (75 lumens, approximately 23° C.) until callus has formed within the graft and new vegetative growth was seen (graft set: approximately 1-2 weeks). New shoot growth was sampled and propagated in tissue culture or soil to isolate individual genome edited lines.

Soil grafting of transgenic hemp lines to wild-type for genome editing was performed consistent with the following protocol. Approximately 2-3 weeks prior to the experiment, hemp wild-type and transgenic plants were propagated in 6-inch pots with organic perlite media (Espoma) saturated with Clonex working solution (Hydrodynamics International: CCS) using shoot cuttings dipped in Hormodin (Olympic Horticultural Products) and sanitized tools. The propagated plants were placed under 16/8-hour light/dark (75 lumens, approximately 23° C.). Once plantlets reached the 5-node stage, fully emerged leaves basal to the 4th node were removed, leaving a single node of fully emerged leaves and the apical meristem. Scion cuttings from the wild-type plantlets were prepared by excising the shoot, abaxial to the 4th node by making 60-degree cuts on either side, creating a spear cut end. Rootstock cuttings were prepared from the transgenic plant by excising the shoot, abaxial to the 2nd node by making a vertical cut in the middle of the cut surface, creating a V-cut while keeping the rootstock in the media. A sterile grafting clip or tape matching the diameter of the cut end of the rootstock so was placed that at least 1 cm of the clip or tape was overlapping on each side of the cut end. The cut end of the scion was inserted into the cut end of the rootstock and secured using the grafting clip or tape. Grafting stakes were used as needed to secure the scion and maintain direct contact with the rootstock. The grafted plant was placed under 16/8-hour light/dark (75 lumens, approximately 23° C.) until callus was formed within the graft and new vegetative growth was seen. New shoot growth was sampled and propagated in tissue culture or soil to isolate individual genome edited lines.

MS media (Phytatrays™ or plates) was prepared using the following protocol, to create 1 L of media:
1600 ml of ddH₂O;
10 g of Sucrose;
4.43 g of MS Basal Salts+Vitamins (Phytotech, M519);
The solution was brought to volume with 1000 ml of ddH₂O;
The pH was adjusted to 5.7 by titration of KOH;
3.58 g of Gelzan™ (Phytotech, G3251);
   The media was autoclaved on liquid cycle for 25 minutes, cooled to 55 C and poured 100 mL per Phytatray™ or 100×25 mm plates.

MMSmedia (Magenta Boxes™) was prepared using the following protocol, to create 1 L of media:
800 ml of ddH₂O;
25 g of Sucrose;
4.43 g of MS Basal Salts+Vitamins (Phytotech, M519);
The solution was brought to volume with 1000 ml of ddH₂O
The pH was adjusted to 5.7 by titration of KOH;
7.5 g of Agar (Phytotech, A296);
The media was autoclaved on liquid cycle for 25 minutes;
0.8 ml of Cefotaxime (250 mg/ml);
0.1 ml of 6-BAP (1 mg/ml);
   The media was cooled to 55 C and poured 100 mL per Magenta Boxes™ (Sigma, V8505).

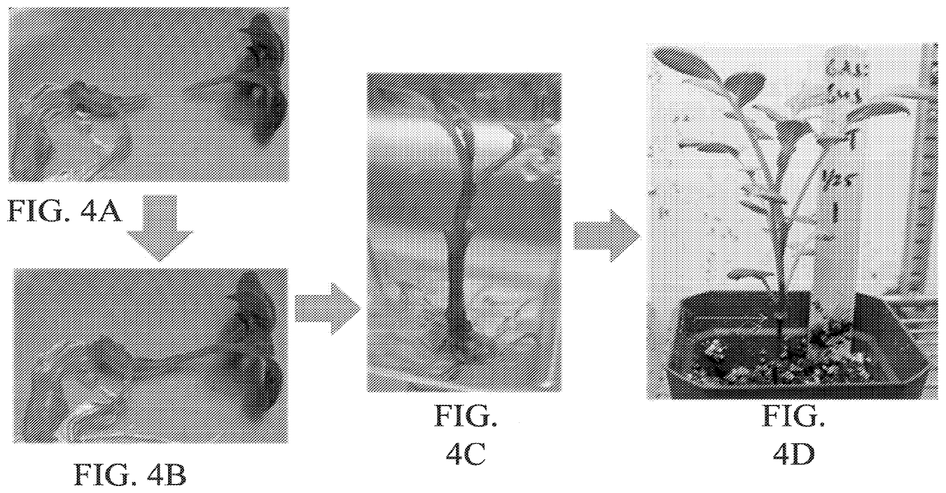

FIGS. 4A, 4B, 4C, and 4D illustrate stages of generating a micrografted potato plant, consistent with the present disclosure. FIG. 4A illustrates the grafted potato plant which includes a transgenic rootstock and a wild-type scion. FIG. 4B illustrates the grafted potato plant with the transgenic rootstock and wild-type scion coupled, as described herein. FIG. 4C illustrates the grafted potato plant 1-2 weeks after grafting, and FIG. 4D illustrates the grafted potato plant 2 weeks after transfer to soil.

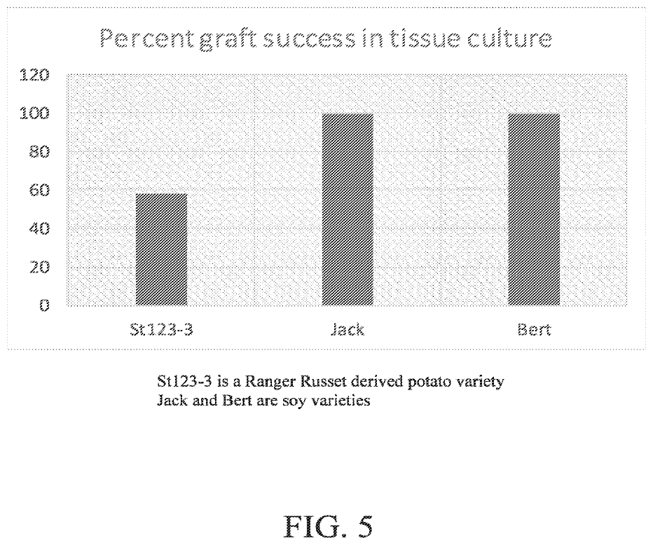

FIG. 5 illustrates data obtained from various grafting experiments conducted, consistent with the present disclosure. Particularly, FIG. 5 illustrates the percent graft success in tissue culture for St123-3 (a Ranger Russet derived potato variety), the Jack variety of soybean, and the Bert variety of soybean. The data illustrates the percent of the respective varieties that were successfully grafted with a transgenic plant, as discussed above. For St123-3 grafts, approximately 60% of all grafted plants continued to propagate new shoot growth, as illustrated in FIG. 4. For Jack grafts, approximately 100% of all grafted plants continued to propagate new shoot growth, as illustrated in FIG. 4. For Bert grafts, approximately 100% of all grafted plants continued to propagate new shoot growth, as illustrated in FIG. 4.

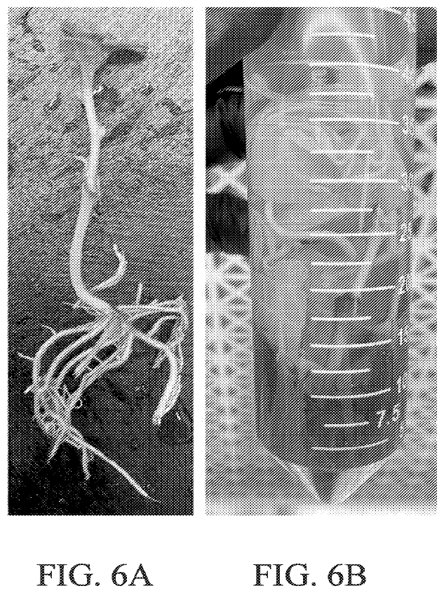

FIGS. 6A and 6B illustrate transgenic gene expression in the rootstock of grafted soy plants, consistent with the present disclosure. Specifically, FIG. 6A illustrates generating grafted soy plants expressing TALEN, as discussed with regards to FIG. 3A. FIG. 6B illustrates a control experiment in which the transgenic rootstock of the grafted soy plants were generated without TALEN and with a GUS marker to indicate movement of the mRNA and/or protein. As illustrated in FIG. 6B, without the TALEN, the marker mRNA and/or protein was consolidated in the transgenic rootstock tissue which resulted in the blue staining in the lower portion of the plant.

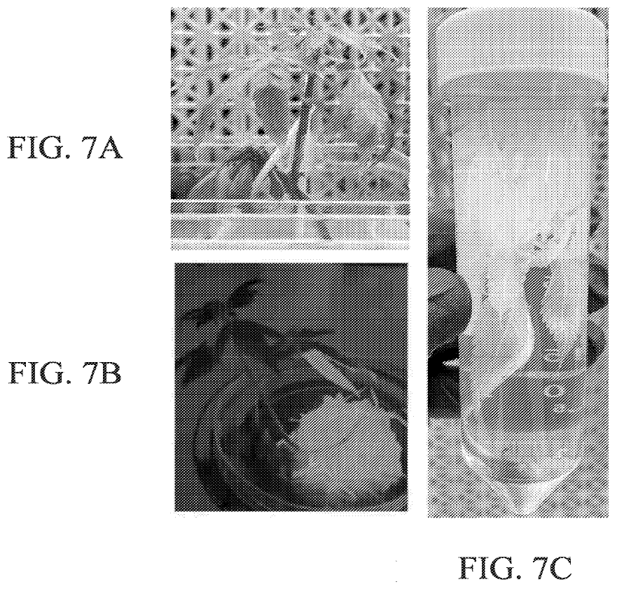

FIGS. 7A, 7B, and 7C illustrate transgenic gene expression in the rootstock of grafted hemp plants, consistent with the present disclosure. Specifically, FIG. 7A illustrates generating micrografted hemp plants using TALEN, as discussed with regards to FIG. 3A. FIG. 7B illustrates yellow fluorescent protein (YFP) expression indicating TALEN expression in transgenic rootstocks of grafted hemp plants. FIG. 7C illustrates a control experiment in which the transgenic rootstock of the grafted hemp plants were generated without the TALEN and with a GUS marker to indicate movement of the mRNA and/or protein. As illustrated in FIG. 7C, without the TALEN, the marker mRNA and/or protein was consolidated in the transgenic rootstock tissue which resulted in the blue staining in the lower portion of the plant.

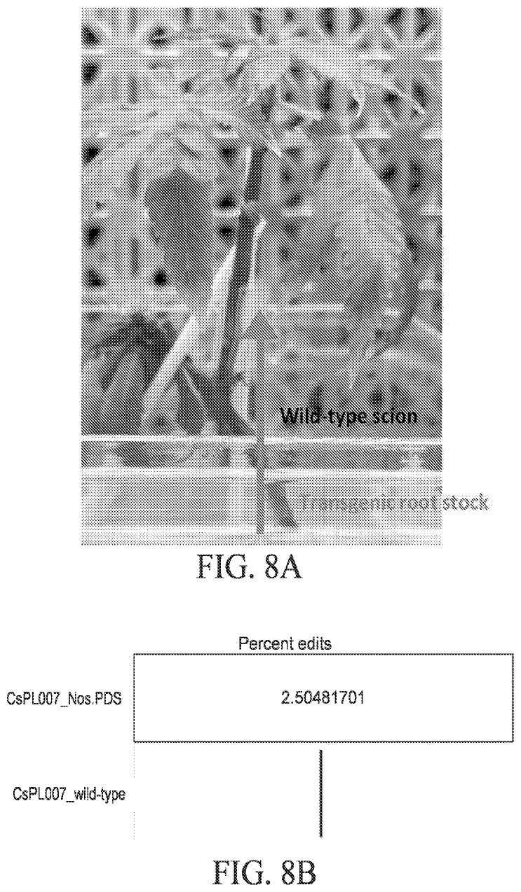

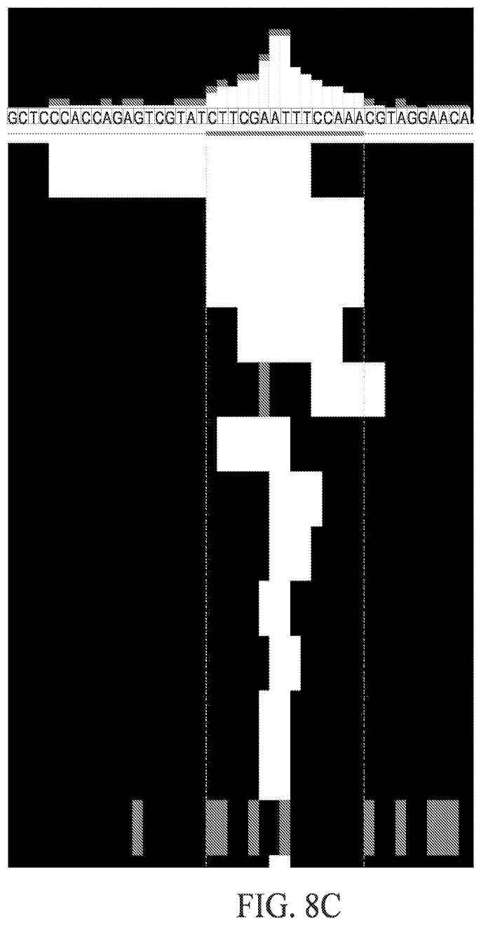

FIGS. 8A, 8B, and 8C illustrate results of genomic editing of wild-type scion tissues in grafted hemp plants, consistent with the present disclosure. FIG. 8A illustrates a grafted hemp plant, with a wild-type scion on the top and a transgenic rootstock on the bottom. FIG. 8B illustrates a comparison of the percent of genomic edits detected in the scion of the grafted hemp plant illustrated in FIG. 8A. Specifically, the bottom half of FIG. 8B illustrates the percent of genomic edits detected in the scion of a wild-type Cannabis sativa control sample, whereas the top half of FIG. 8B illustrates the percent of gene edits detected in the grafted Cannabis sativa plant illustrated in FIG. 8A. The transgenic rootstock included a Nos promoter and a TALEN mRNA sequence targeting the CsPDS gene. As illustrated in FIG. 8B, the grafted Cannabis sativa demonstrated 2.50 percent edits whereas the wild-type Cannabis sativa did not demonstrate any edits. These results are further illustrated in FIG. 8C, which illustrates genomic edits detected by Illumina® amplicon sequencing reads obtained from scion tissues of the grafted plant illustrated in FIG. 8A.

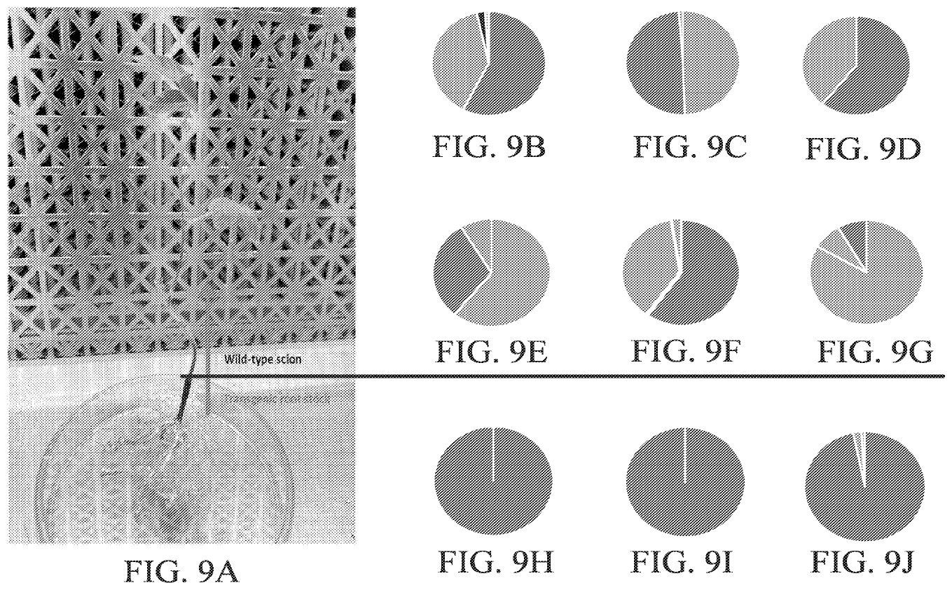

FIGS. 9A, 9B, 9C, 9D, 9E, 9F, 9G, 9H, 9I, and 9J illustrate results of genomic editing in tissues of grafted soy plants, consistent with the present disclosure. FIG. 9A illustrates a grafted soy plant, with a wild-type scion (Jack or Bert) and transgenic rootstock (Gm1559) generated as described herein. FIGS. 9H, 9I, and 9J illustrate the amount and composition of genomic edits present in control tissue of three FAD3 genes (FAD3a, FAD3b and FAD3c). Specifically, FIG. 9H illustrates the composition of genomic edits present in the FAD3a gene of the Gm1559 transgenic control, FIG. 9I illustrates the composition of genomic edits present in the FAD3b gene of the Gm1559 transgenic control, and FIG. 9J illustrates the composition of genomic edits present in the FAD3c gene of the Gm1559 transgenic control. In FIGS. 9H and 9I, the transgenic controls include genomic edits represented by the blue composition of the pie graph. FIG. 9J illustrates the genomic edits in the transgenic control, represented by the blue, orange, and grey portions of the pie graph.

FIGS. 9B, 9C, 9D, 9E, 9F, and 9G illustrate the composition of genomic edits detected in the wild-type scion of the grafted plant, as illustrated in FIG. 9A. In particular, FIG. 9B illustrates the genomic edits detected in the FAD3a gene when a Jack variety of soybean was grafted to the Gm1559 transgenic rootstock The result was that the genomic edits represented in FIG. 9H (e.g., the blue portion of the pie chart) was detected in the wild-type scion, but additional genomic edits were detected, represented by the green, black, and yellow portions of the pie chart illustrated in FIG. 9B. Similarly, FIG. 9C illustrates the genomic edits detected in the FAD3b gene when the Jack variety of soybean was grafted to the Gm1559 transgenic rootstock. The result was that the genomic edits represented in FIG. 9I (e.g., the blue portion of the pie chart) was detected in the wild-type scion, but additional genomic edits were detected, represented by the green, and grey portions of the pie chart illustrated in FIG. 9C. Similarly, FIG. 9D illustrates the genomic edits detected in the FAD3c gene when the Jack variety of soybean was grafted to the Gm1559 transgenic rootstock. The result was that portions of the genomic edits represented in FIG. 9J (e.g., the blue, orange, and grey portions of the pie chart) were detected in the wild-type scion, represented by the blue and grey portions of the pie chart illustrated in FIG. 9D, but at differing amounts.

FIG. 9E illustrates the genomic edits detected in the FAD3a gene when a Bert variety of soybean was grafted to the Gm1559 transgenic rootstock. The result was that the genomic edits represented in FIG. 9H (e.g., the blue portion of the pie chart) was detected in the wild-type scion, but additional genomic edits were detected, represented by the green and grey portions of the pie chart illustrated in FIG. 9E. Similarly, FIG. 9F illustrates the genomic edits detected in the FAD3b gene when the Bert variety of soybean was grafted to the Gm1559 transgenic rootstock. The result was that the genomic edits represented in FIG. 9I (e.g., the blue portion of the pie chart) was detected in the wild-type scion, but additional genomic edits were detected, represented by the green and grey portions of the pie chart illustrated in FIG. 9F. Similarly, FIG. 9G illustrates the genomic edits detected in the FAD3c gene when the Bert variety of soybean was grafted to the Gm1559 transgenic. The result was that portions of the genomic edits represented in FIG. 9J (e.g., the blue, orange, and grey portions of the pie chart) were detected in the wild-type scion, but additional genomic edits were detected, represented by the green portions of the pie chart illustrated in FIG. 9G.

1. A method for producing a grafted plant for delivery of genome editing reagents, comprising:
grading cultured rootstock tissue to obtain at least one rootstock expressing an expression construct and having a stem with a graft-compatible diameter and genotype;
making a cut through the at least one rootstock stem and placing a stabilization device adjacent to the cut on the rootstock stem;
generating a grafted plant by inserting at least one cut scion stem into the stabilization device, wherein the at least one cut scion stem substantially aligns with vascular tissue within the cut rootstock stem; and
screening new growth from the grafted plant for gene edits resulting from genomic editing by the expression construct.

2. The method of claim 1, including generating a transgenic plant expressing the expression construct in a genotype that is graft-compatible with a genotype of the cut scion stem.

3. The method of claim 1, including generating a transgenic plant expressing the expression construct by infecting a host plant with Agrobacterium tumefaciens carrying the expression construct.

4. The method of claim 1, including generating a transgenic plant expressing the expression construct by infecting a host plant with Rhizobium rhizogenes carrying the expression construct.

5. The method of claim 1, including generating a transgenic plant expressing the expression construct using particle bombardment.

6. The method of claim 1, wherein making the cut through the at least one rootstock stem includes making a first angled cut through the at least one rootstock stem, the method further including making an second angled cut through the at least one scion stem, wherein the second angled cut through the at least one scion stem is substantially similar to the first angled cut through the at least one rootstock stem.

7. The method of claim 1, wherein making the cut through the at least one rootstock stem includes making a first wedge-shaped cut through the at least one rootstock stem, the method further including making a second wedge-shaped cut through the at least one scion stem, wherein the second wedge-shaped cut through the at least one scion stem is substantially similar to the first wedge-shaped cut through the at least one rootstock stem.

8. The method of claim 1, wherein the expression construct includes transcription activator like effector nuclease (TALEN) mRNA, and wherein screening the new growth from the grafted plant includes sampling new shoot growth for the TALEN mRNA and/or protein using end-point reverse transcriptase PCR (RT-PCR) or western blot.

9. The method of claim 1, wherein the expression construct includes:
an mRNA coding sequence; and
a promoter.

10. The method of claim 9, wherein the promoter is 35S.

11. The method of claim 9, wherein the promoter is nopaline synthase (Nos).

12. A non-naturally occurring plant, plant cell, or plant part generated by a genomic editing technique comprising:
generating a transgenic plant by infecting a host plant with Agrobacterium tumefaciens solution including:
a transcription activator like effector nuclease (TALEN) messenger ribonucleic acid (mRNA) coding sequence; and
a promoter;
grading rootstock tissue of the transgenic plant to obtain at least one rootstock expressing an expression construct and having a stem with a graft-compatible diameter; and
generating a grafted plant by inserting at least one cut scion stem into the stabilization device, wherein the at least one cut scion stem substantially aligns with vascular tissue within the cut rootstock stem.

13. The non-naturally occurring plant of claim 12, wherein the genomic editing technique includes making a cut through the at least one rootstock stem and placing a stabilization device adjacent to the cut on the rootstock stem.

14. The non-naturally occurring plant of claim 12, wherein the genomic editing technique includes screening new growth from the grafted plant for gene edits resulting from genomic editing by the expression construct.

15. The non-naturally occurring plant of claim 12, wherein the mRNA coding sequence includes a Glycine max fatty-acid-desaturase 3 (GmFAD3) gene, a Cannabis sativa phytoene desaturase (CsPDS) gene, or a Solanum tuberosum phytoene desaturase (StPDS) gene.
